(19) United States
(12) Patent Application Publication
(10) Pub. No.: US 2025/0349415 A1
(43) Pub. Date: Nov. 13, 2025

(54) ANALYSIS OF THREE-DIMENSIONAL PATHOLOGY SAMPLES USING ARTIFICIAL INTELLIGENCE

(71) Applicant: THE BRIGHAM AND WOMEN'S HOSPITAL, INC., Boston, MA (US)

(72) Inventors: Faisal Mahmood, Brookline, MA (US); Andrew H. Song, Boston, MA (US)

(21) Appl. No.: 19/203,297

(22) Filed: May 9, 2025

Related U.S. Application Data
(60) Provisional application No. 63/645,051, filed on May 9, 2024.

(51) Int. Cl. G16H 30/40; G06N 20/00; G16H 50/20; G16H 50/70

(52) U.S. Cl. CPC G16H 30/40; G06N 20/00; G16H 50/20; G16H 50/70

(57) ABSTRACT

Determining a patient-level clinical endpoint prediction based on analysis of a three-dimensional volumetric image is discussed. One example method includes generating a set of patches from a volumetric image of a tissue sample. The method also includes employing a pretrained feature encoder to extract a set of features from the set of patches. The method additionally includes generating a volume-level feature associated with the volumetric image via an aggregation based on the set of features. The method further includes generating a clinical endpoint prediction based on the volume-level feature.

### RELATED APPLICATION

This application claims the benefit of U.S. Provisional Patent Application Ser. No. 63/645,051, filed May 9, 2024, the entirety of which is hereby incorporated by reference for all purposes.

### BACKGROUND

Histopathology is the analysis (e.g., via microscopy, etc.) of tissue samples, for example, to determine a diagnosis, prognosis, etc. Human tissue, which is inherently three-dimensional (3D), is traditionally examined through standard-of-care histopathology as limited two-dimensional (2D) cross-sections. For example, one or more selected stained histological slides from a tissue sample (e.g., biopsy, etc.) are examined under a microscope by a trained pathologist to determine characteristics based on the tissue (e.g., a diagnosis, prognosis, etc.).

### SUMMARY

A first example relates to a non-transitory machine-readable medium having machine executable instructions for a physiological signal reconstruction system that cause a processor core to execute operations. The operations include generating a set of patches from a volumetric image of a tissue sample. The operations also include employing a pretrained feature encoder to extract a set of features from the set of patches. The operations additionally include generating a volume-level feature associated with the volumetric image via an aggregation based on the set of features. The operations further include generating a clinical endpoint prediction based on the volume-level feature.

A second example relates to a non-transitory machine-readable medium having machine executable instructions for training a physiological signal reconstruction system that cause a processor core to execute operations. The operations include accessing a training set including a set of volumetric images and a set of ground-truth clinical endpoints. The operations also include generating patches from the set of volumetric images. An associated set of patches is generated from a volumetric image of the set of volumetric images. The operations additionally include employing a pretrained feature encoder to extract features from the patches. An associated set of features is extracted from the associated set of patches. The operations further include generating a set of volume-level features associated with the set of volumetric images via an aggregation based on the features. An associated volume-level feature of the set of volume-level features is generated based on the associated set of features. Additionally, the operations include training an artificial intelligence (AI) model based on the set of volume-level features and the set of ground-truth clinical endpoints. The AI model is trained based on the associated volume-level feature and a ground-truth clinical endpoint of the set of ground truth-clinical endpoints, and the ground-truth clinical endpoint is associated with the volumetric image.

A third example relates to a method that includes generating a set of patches from a volumetric image of a tissue sample. The method also includes employing a pretrained feature encoder to extract a set of features from the set of patches. The method additionally includes generating a volume-level feature associated with the volumetric image via an aggregation based on the set of features. The method further includes generating a clinical endpoint prediction based on the volume-level feature.

### BRIEF DESCRIPTION OF THE DRAWINGS

FIG. 1 illustrates an example computing environment implementing a three-dimensional (3D) pathology prediction system capable of generating a clinical endpoint prediction based on input volumetric image(s) of a tissue sample.

FIG. 2 illustrates example steps involved in acquisition of a volumetric image according to two different 3D imaging techniques.

FIG. 3 illustrates example steps involved in processing (a volumetric image to generate a set of patches for analysis.

FIG. 4 illustrates example steps involved in analyzing a set of patches to generate a clinical endpoint prediction.

FIG. 5 illustrates a flow diagram of a method for generating a clinical endpoint prediction based on a volumetric image of a tissue sample.

FIG. 6 illustrates a flow diagram of a method for training an artificial intelligence (AI) model to generate a clinical endpoint prediction based on a volumetric image of a tissue sample.

### DETAILED DESCRIPTION

Various example systems and methods described herein provide techniques for employing an artificial intelligence (AI) model to generate clinical endpoint prediction(s) based on analysis of volumetric image(s) of tissue sample(s). Examples employ deep learning to process the volumetric image(s) and efficiently predict clinical endpoints (e.g., diagnoses, prognoses, etc.) based on three-dimensional (3D) morphological features.

Human tissues are collections of diverse heterogeneous structures that are intrinsically 3D. Despite this, the examination of thin two-dimensional (2D) tissue sections mounted on glass slides has been the diagnostic standard for over a century. Tissue sampling in 2D represents only a small fraction of the complex morphological information inherent in all three dimensions. For example, it has been shown that diagnoses are more accurate in certain applications when multiple levels are examined from the same tissue block instead of a single 2D slice. Furthermore, certain characteristics of complex tissue micro-structures are ambiguous or entirely opaque in 2D cross-sectional histology images. Accordingly, various examples facilitate 3D pathology, allowing for better characterization of the morphological diversity encountered in an entire tissue volume, improving predictions such as patient diagnosis, prognosis, prediction of treatment response, biomarker discovery to facilitate companion diagnostics, etc.

Several 3D imaging techniques have emerged over the past decade to holistically capture volumetric tissue morphologies. In addition to protocols for serial sectioning of tissue followed by 3D reconstruction, non-destructive imaging modalities such as high-throughput 3D light-sheet microscopy (e.g., open top light-sheet microscopy (OTLS), etc.), microcomputed tomography (microCT), photoacoustic microscopy, multiphoton microscopy, and optical coherence tomography are also useable for capturing high-resolution 3D volumetric tissue images. However, several barriers to the clinical adoption of 3D imaging techniques still exist. One of the predominant challenges is to efficiently and accurately analyze the large, feature-rich 3D datasets that these techniques routinely generate. The addition of the depth dimension can increase the size of high-resolution histology images by several orders of magnitude and render manual examination of tissue by pathologists, a workflow that can already be tedious in 2D, even more time-consuming and error-prone without assistance in analyzing the data.

Various examples employ computational approaches based on deep learning (DL) for analyzing large 3D pathology datasets, providing diagnostic determinations and decision support efficiently and automatically. Existing DL-based computational pathology frameworks are based on 2D tissue images, utilize hand-engineered 3D features, and/or are based on predefined morphometric descriptors that are limited in scope and involve sophisticated segmentation networks to first delineate selected tissue primitives. In contrast, various examples provide an end-to-end DL approach capable of identifying novel visual features based on a clinical endpoint in an unconstrained fashion with the potential to maximize analytical performance.

Examples employ a DL-based computational pipeline for volumetric image analysis that can perform patient prognostication based on 3D morphological features based on patient-level clinical endpoint labels without the need for manual annotations by pathologists. Examples are useable as a general-purpose computational tool for tissue volume analysis, which is agnostic towards imaging modality and can be flexibly adapted for 2D and 3D analyses of volumetric inputs to cater to diverse tasks.

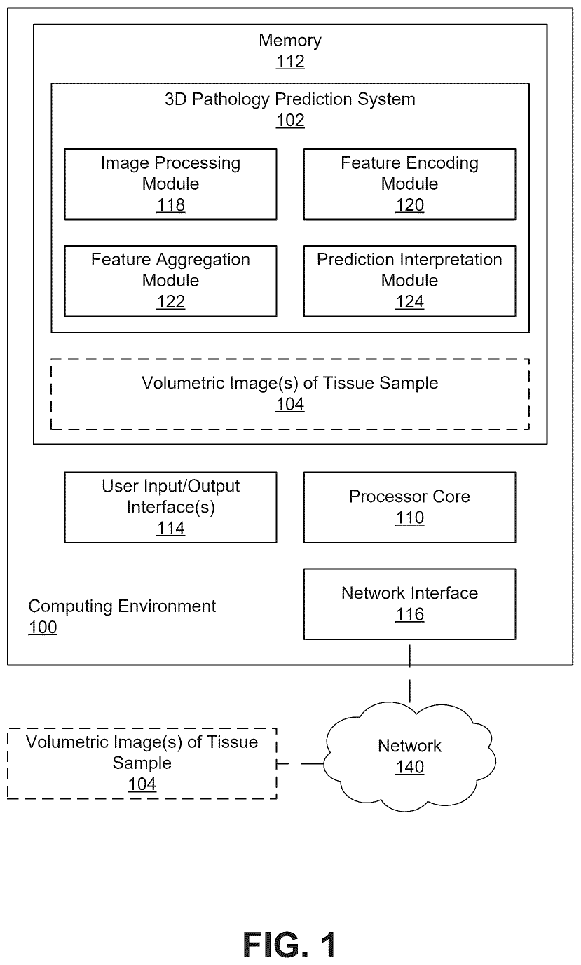

FIG. 1 illustrates an example computing environment 100 implementing a three-dimensional (3D) pathology prediction system 102 capable of generating a clinical endpoint (e.g., patient-level, etc.) prediction based on one or more input volumetric images 104 of a tissue sample. The 3D pathology prediction system 102 encodes a set of features (e.g., as a set of vectors, etc.) from a set of patches determined from a volumetric image(s) 104 of the tissue sample, aggregates the set of features (or a set of compressed features determined from the set of features) to generate a volume-level feature, and generates a patient-level prediction based on the volume-level feature. In various examples, the volumetric image(s) 104 of the tissue sample are stored locally at the computing environment 100 (e.g., as shown within the memory 112, etc.) and/or remotely (e.g., as shown connected to the computing environment 100 via the network 140).

The computing environment 100 includes a processor core 110, a memory 112, a user input/output (I/O) interface 114, and a network interface 116, which are operably connected for computer communication. The processor core 110 performs general computing to execute instructions stored in the memory 112, including instructions associated with the 3D pathology prediction system 102. The instructions cause the processor core 110 to execute operations. The memory 112 also stores instructions associated with an operating system that controls and/or allocates resources of computing environment 100, including resources associated with the 3D pathology prediction system 102. Memory 112 represents a non-transitory machine-readable memory (or other medium), such as random access memory (RAM), a solid state drive, a hard disk drive or a combination thereof.

Processor core 110 accesses memory 112 and executes the machine-readable instructions as operations. Processor core 110 can be a variety of various processors including multiple single- and multi-core processors, co-processors, and other multiple single and multicore processor and co-processor architectures.

User I/O interface 114 provides software and hardware to facilitate data input and output between computing environment 100 and a user. This can include input devices such as a keyboard, mouse, touchpad, touchscreen, microphone, camera, etc., as well as output devices such as display(s) (e.g., light-emitting diode (LED) display panel(s), liquid crystal display (LCD) panel(s), plasma display panel(s), and/or touch screen display(s), etc.), speaker(s), etc. User I/O interface 114 provides graphical input controls for a user interface, which can include software and hardware-based controls, interfaces, touch screens, or touch pads or plug and play devices for a user to provide user input.

The memory 112 includes the 3D pathology prediction system 102, which includes one or more of an image processing module 118, a feature encoding module 120, a feature aggregation module 122, and a prediction interpretation module 124 that operate in concert and/or stages to generate a patient-level prediction based on analysis of volumetric image(s) 104 of the tissue sample. In various examples, the 3D pathology prediction system 102 determines a set of patches from pre-processed (e.g., by the 3D pathology prediction system 102, etc.) volumetric image(s) 104 of the tissue sample and generates associated features for each patch via a pretrained feature encoder network (e.g., pretrained on similar volumetric images and/or any of a variety of images or videos, such as histopathology images, natural images, 3D medical imaging datasets, human action recognition videos, etc.). The 3D pathology prediction system 102 aggregates the set of features associated with the set of patches (e.g., based on weightings associated with the importance of features towards contributing to a volume level feature to render a clinical endpoint prediction, etc.) to determine a volume level feature that is used to generate a clinical endpoint prediction.

In various examples, the 3D pathology prediction system 102 divides an input volumetric image 104 (e.g., which can have, e.g., >109 voxels, etc.) into a set of patches with smaller volumes, which are then summarized into a set of features that can be expressed as a single low-dimensional feature vector (e.g., which can have a size based on the number of patches, e.g., on the order of ˜ 103 for the example input volume size, etc.). Various examples utilize the set of features (e.g., feature vector, etc.) as the basis for generating a patient-level clinical endpoint prediction. In some examples, n input volumetric images 104 (e.g., obtained via different imaging modalities of the same tissue sample, etc.) are used to generate associated subsets of patches that are summarized into a set of features (e.g., n associated subsets of features, n feature vectors, and/or a single concatenated feature vector, etc.) that are used as the basis for determining the endpoint prediction.

The image processing module 118 generates a set of patches from the volumetric image(s) 104 (e.g., including a subset of patches for each volumetric image of the volumetric image(s), etc.). In some examples, the image processing component 118 segments a given volumetric image 104 into a set of sub-volumes (e.g., a stack of planes (2D), cuboids (3D), etc.) that contain tissue (e.g., pre-segmented in the volumetric image 104 or segmented via the image processing module 118, etc.) and further tessellates the sub-volumes into smaller 2D or 3D patches, which allows for direct computational processing.

In various examples, the feature encoding module 120 compresses extracted features from each patch of the set (e.g., with a pretrained feature encoder, such as a pretrained 2D or 3D DL feature encoder, and a feedforward network, such as a task-adaptable shallow feedforward network, etc.) to generate a set of features (e.g., feature vector, etc.). The feature aggregation module 122 weights and aggregates the set of features associated with the set of patches to form a volume-level feature for patient-level risk prediction (e.g., via generation of a patient-level clinical endpoint prediction by the feature aggregation module 122, etc.). In various examples, the feature aggregation module 122 employs an attention-based aggregation module to automatically identify important patches and regions contributing to prognostic decisions without additional pathologist annotations. Additionally, in various examples, as a post-hoc interpretation method, the prediction interpretation module 124 generates additional information indicating features and/or morphology associated with the endpoint prediction, for example, via one or more interpretability techniques such as saliency heatmap(s) for the network prediction, which are useable to identify intuitive morphological correlated with the clinical endpoints (e.g., with coloring based on saliency values such as integrated gradient (IG) attribution scores, etc.), by visually representing the importance of regions of the volumetric image to the clinical endpoint prediction. In various examples, regions of the volumetric image(s) 104 identified as high risk or otherwise significantly contributing (e.g., via false coloring based on saliency values (e.g., IG scores, etc.), by identifying region(s) with saliency value (e.g., IG score, etc.) above or below a selected threshold and/or in a selected top or bottom percentile of saliency values (e.g., IG scores), etc.) to the patient-level prediction are identified by the prediction interpretation module 124, allowing for further evaluation by a user (e.g., pathologist, etc.).

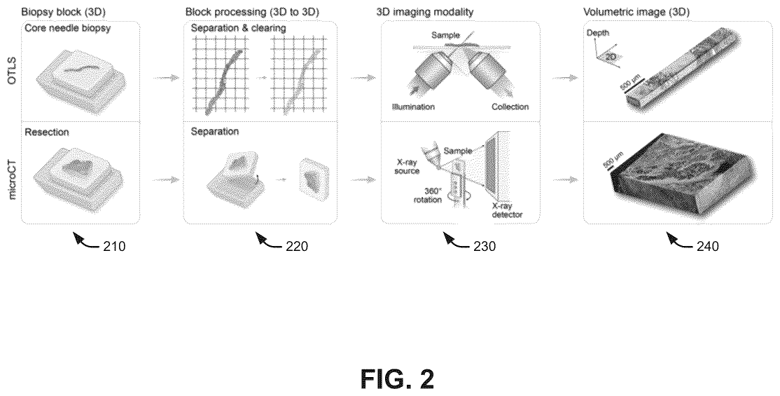

FIG. 2 illustrates example steps 210-240 involved in acquisition of a volumetric image (e.g., of the volumetric image(s) 104, etc.) according to two different 3D imaging techniques, OTLS (top row) and microCT (bottom row). While OTLS and microCT are shown as examples of 3D imaging techniques, various examples are employable for generating predictions based on volumetric images obtained via any of a variety of existing or not yet developed 3D imaging techniques, including photoacoustic microscopy, multiphoton microscopy, optical coherence tomography, holotomography, etc. At 210, a 3D biopsy block is obtained and/or accessed, where the biopsy block can depend on the 3D imaging technique to be used (e.g., a core needle biopsy for OTLS, a tissue resection for microCT, etc.). At 220, the biopsy block is processed to generate a sample for subsequent imaging, such as separation and cleaning for OTLS or separation for microCT. At 230, 3D imaging of the selected imaging modality is applied to the sample, such as illumination of the sample and associated light collection for OTLS, or repeated transmission of x-rays from an x-ray source through the sample to an x-ray detector as the sample is incrementally rotated (e.g., through 360°, 180°, etc.). At 240, the data collected via the imaging at 230 is reconstructed into the 3D volumetric image.

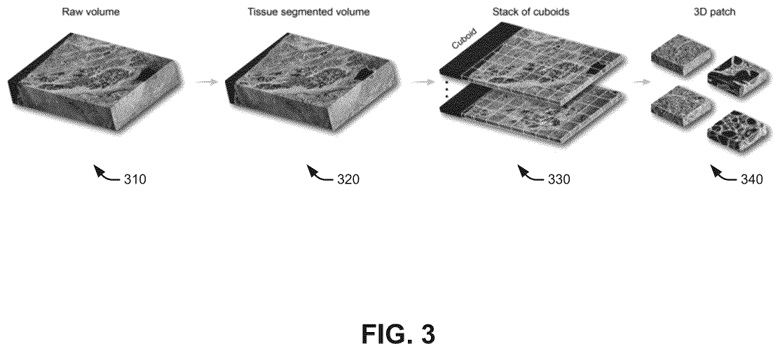

FIG. 3 illustrates example steps 310-340 involved in processing (e.g., via the image processing module 118, etc.) a volumetric image (e.g., of the volumetric image(s) 104, etc.) to generate a set of patches for analysis. At 310, a raw volumetric image (e.g., generated via steps 210-240 of FIG. 2, etc.) is accessed. At 320, tissue segmentation is performed to identify portions of the volumetric image with tissue and generate a segmented volumetric image that excludes non-tissue portions of the raw volumetric image from analysis. Steps 330 and 340 show one example technique of generating patches for analysis from the segmented volumetric image. At 330, the segmented volumetric image is treated as a stack of cuboids (e.g., rectangular cuboids, etc.) that are tessellated at 340 into a set of patches (e.g., 3D patches as shown at 340, 2D patches, etc.).

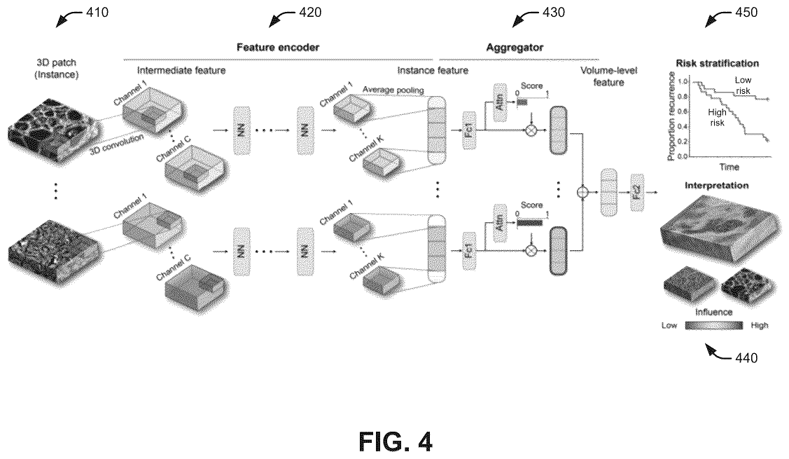

FIG. 4 illustrates example steps 410-440 involved in analyzing a set of patches to generate a clinical endpoint prediction. At 410, a set of patches (e.g., the 3D patches shown at 340 in FIG. 3, etc.) of a volumetric image (e.g., of the volumetric image(s) 104, etc.) are accessed. At 420, the set of patches are processed (e.g., via the feature encoding module 120, etc.) with a pretrained feature encoder network (with a set of neural network layers NN that depend on the selected feature encoder network, etc.) that can vary between examples (e.g., a 2D/3D CNN as used in FIG. 4, a 2D/3D Vision Transformer, etc.), leveraging transfer learning to produce a set of compact and representative features, which are compressed to instance features (e.g., via a domain-adapted shallow, fully-connected network as in FIG. 4, etc.). At 430, an aggregator module (e.g., the feature aggregation module 122, etc.) aggregates the set of features representing all instances, automatically weighting them according to their importance towards contributing to a volume-level feature (e.g., via fully connected layer Fc1 and attention module Attn in FIG. 4, etc.) used to render a patient-level prediction (e.g., via fully connected layer Fc2 in FIG. 4, etc.). At 440, saliency heatmaps are generated for clinical interpretation and/or validation based on the importance of various patches toward contributing to the volume-level feature. Additionally, FIG. 4 at 450 shows the proportion of patients experiencing disease recurrence over time for low risk and high risk groups in connection with a prototype example.

Volume-based 3D analysis according to various examples provides multiple advantages over 2D techniques. From a clinical perspective, various examples reliably include prognostically important regions not present in traditional whole slide images (WSIs), which have limited coverage of morphologically heterogeneous tissue. In addition to 2D-based architectures pretrained on 2D natural images, various examples employ 3D feature encoder(s) (e.g., 3D convolutional neural networks (CNN) and/or 3D Vision Transformers (ViT), etc.) pretrained on image sequences to encode 3D-morphology-aware low-dimensional features from patches (e.g., 2D or 3D patches, etc.). Unlike techniques that are based on hand-engineered features that are limited by human cognition and involve sophisticated segmentation networks to delineate specific tissue primitives, an especially challenging task in 3D, various examples employ automatic encoding of morphological representations with a DL-based feature encoder.

Examples provide multiple advantages over existing techniques. Compared to WSI analysis, various examples utilizing the entire 3D volume eliminate the sampling bias involved in slide selection for WSIs and the probability of missing slides that strongly affect the predicted clinical endpoint. The majority of medical imaging applications rely on the identification and segmentation of specific morphologies, involving pixel-level or slice-level annotations. In contrast, various examples determine patient-level labels (e.g., clinical endpoints, etc.) without manual annotations by clinicians. Moreover, existing 3D medical imaging frameworks deal with lower-resolution images (>1 mm/voxel) and much smaller datasets (roughly a sequence of 100 images of at most 512×512 pixels) compared to the gigavoxel 3D pathology scans (˜1 μm/voxel) analyzed by various examples. As a result, existing medical imaging techniques are inapplicable to 3D pathology, unlike various examples. Additionally, examples are agnostic towards input modalities and components such as feature encoders, allowing the same examples to be employed with a variety of existing or future architectures (e.g., Transformer, hierarchical-aggregation-based, etc.) or imaging modalities.

Prototype examples were tested in various contexts, including a classification task with simulated 3D phantom datasets and prognostication tasks for two different prostate cancer cohorts imaged with different 3D imaging modalities (e.g., with a clinical endpoint of the duration between prostatectomy and the occurrence of biochemical recurrence (BCR), marked by an elevation in prostate-specific antigen (PSA) levels surpassing a defined threshold). The prototypes compared several analytical treatments of the volumetric samples, from utilizing 2D patches from a few planes within each volume (emulating a traditional 2D pathology workflow) to utilizing 3D patches from the whole volume. Different prototypes were trained for multiple imaging modalities (e.g., OTLS, microCT, etc.) and tested on both the trained imaging modality and other imaging modalities (e.g., an OTLS-trained prototype tested on microCT, a microCT-trained prototype tested on microCT, etc.). Various example prototypes outperformed clinical baseline testing (e.g., based on WSIs, etc.), with the whole volume approaches and 3D features encoded from 3D patches providing the highest performance as measured by the area under the receiver operating curve (AUC).

Additionally, various examples employ (e.g., via the prediction interpretation module 124, etc.) saliency analysis (e.g., an integrated gradient (IG) analysis, etc.) wherein a saliency value (e.g., an IG attribution score, etc.) is computed for each patch in connection with the prediction (e.g., generated by the feature aggregation module 122, etc.). Positive (high) scores are associated with regions increasing the predicted risk (e.g., unfavorable prognosis, etc.), while negative (low) scores are associated with regions that decrease the predicted risk (e.g., favorable prognosis, etc.). In various examples, the saliency (e.g., IG, etc.) values are overlaid on the raw volume input (e.g., the volumetric image 104, etc.) to generate (e.g., via the prediction interpretation module 124, etc.) saliency (e.g., IG, etc.) interpretability heatmaps and to further locate regions of different prognostic information within the tissue volume. In the prototypes trained for prostate cancer prognostication, saliency was evaluated using IG scores and the mean IG score correlated with the predicted risk, while in other examples, the mean saliency value (e.g., IG score, etc.) can correlate with other prediction outcomes (e.g., in connection with a diagnosis, prognosis, biomarker discovery, drug response, etc.).

In various examples, volumetric image(s) (e.g., volumetric image(s) 104, etc.) are pre-processed (e.g., via the image processing module 118, etc.) by performing tissue segmentation. As one example, the volumetric image is treated as a stack of 2D images and tissue segmentation serially on the stack. The mean voxel intensity is computed (e.g., via the image processing module 118, etc.) for each image to identify a subset of stacks containing air and images below a selected (e.g., user-defined, etc.) threshold (e.g., via the image processing module 118, etc.) are disregarded before segmentation. Images in the remaining stack are then converted to grayscale color space, median-blurred to suppress edge artifacts, and binarized with modality-specific thresholds (e.g., via the image processing module 118, etc.). The tissue contours are identified (e.g., via the image processing module 118, etc.) based on the binarized images, and the stack of tissue contours serves as the contour for the volume input. In various examples, images with tissue area below a selected threshold are removed (e.g., via the image processing module 118, etc.) to ensure sufficient tissue exists in each image.

In various examples, the segmented (e.g., pre-segmented or by the image processing module 118, etc.) volumetric image is patched (e.g., via the image processing module 118, etc.) into a set of smaller 2D patches (from a stack of planes) or 3D patches (from a stack of cuboids) to facilitate direct computational processing of the volumetric image. The patch size and the overlap between the patches are chosen in various examples to ensure that context is sufficiently covered within each patch and enough patches exist along each dimension. As one example (of an OTLS volumetric image used in connection with a prototype), a 3D patch size of 128×128×64 voxels (˜128×128×64 μm) was used. An overlap (e.g., of 32 voxels in the example, etc.) along the depth dimension is used in some examples to ensure that enough patches exist along the depth dimension, depending on the size of the volumetric image (e.g., the example segmented a volumetric image with a depth of 320 voxels, etc.). As another example (of a microCT volumetric image used in connection with a prototype), a 3D patch size of 128× 128×32 voxels (˜512×512×128 μm) without any overlap was used as the size of the tissue allowed a sufficient number of patches along all dimensions. For 2D patch, example prototypes used a non-overlapping patch of 128×128 pixels (˜128×128 μm for OTLS and 512×512 μm for microCT) for both modalities. In various examples, greater or smaller patch sizes are useable for 2D and/or 3D patches.

For 3D patching, a reference plane is used in various examples from which the patching operation along the depth dimension is started (e.g., via the image processing module 118, etc.). In various examples, the largest plane by tissue area (identified by the tissue contour from the volume segmentation step) is used as the reference and the two-dimensional patch coordinates within the tissue contour are computed (e.g., via the image processing module 118, etc.). 3D patching in various examples is performed (e.g., via the image processing module 118, etc.) along both directions of the depth dimension starting from the reference plane. The collection of two-dimensional coordinates computed in the reference plane is utilized (e.g., by the image processing module 118, etc.) across the entire volume. Upon completion, in various examples, 3D patches are removed (e.g., via the image processing module 118, etc.) if more than a threshold portion (e.g., 50%, etc.) of the volume (area) constitutes the background to ensure each patch contains sufficient tissue.

In various examples, after patching, the intensity in each patch is clipped at modality-specific lower and upper thresholds and then normalized to [0, 1] for feature encoding (e.g., via the image processing module 118, etc.). In one microCT example, the lower threshold was set to 25,000 intensity value and the upper threshold to the top 1% of the intensity value of the volumetric image of the tissue. In one OTLS example, the lower threshold was set to 100, and the upper threshold to the top 1% of the intensity value of the volumetric image of the tissue. Additionally, in various examples, for OTLS, the normalized intensity values are inverted.

Feature encoder(s) are employed in various examples (e.g., by the feature encoding module 120, etc.) for extracting and encoding a compressed and representative descriptor hⱼ∈ [symbol] ^{K}, j=1, . . . , J of the patch input xⱼ∈ [symbol] ^{L×D×H×W} (3D patch) or xⱼ∈ [symbol] ^{L×D×H×W} (2D patch), where K corresponds to the encoded feature dimension, J denotes the number of patches, L denotes the number of input channels, and D, H, and W denote the depth, height, and width dimension, respectively. Various examples employ (e.g., via the feature encoding module 120, etc.) a range of 2D and 3D pretrained feature encoders based on convolutional neural networks (CNN) or Vision Transformer (ViT) for transfer learning. Example 3D feature encoders employed include a spatiotemporal CNN pretrained on a large collection of human action recognition videos and a video sliding-window transformer (Video SwinViT) pretrained on a human action recognition videos or 3D medical imaging dataset. Example 2D feature encoders employed include a deep residual CNN (e.g., ResNet-50, etc.) pretrained on natural images and SwinViT pretrained on a large collection of histopathology images or natural images.

Due to the scarcity of patient-level labels (clinical endpoints) for 3D pathology datasets and generally larger encoder network size for processing the depth dimension, in various examples a fully-connected linear layer is applied (e.g., via the feature encoding module 120, etc.) to the feature encoder outputs

$${\{ {h}_{j} \}}_{j = 1}^{J} ,$$

parameterized by W_{enc}∈ [symbol] ^{K′×K} and b_{enc}∈R^{K′} (where K′<K is the compressed feature dimension, for example, with K=1024 and K′=256 in prototypes, although greater or lesser values are used in some examples) followed by Gaussian Error Linear Unit (GeLU) nonlinearity. This further converts the patch feature h; from the feature encoder to a more-compressed and domain-specific feature zⱼ∈ [symbol] ^{C} conducive to downstream tasks with better generalization performance, as in equation (1):

$${z}_{j} = GeLU ( {W}_{enc} {h}_{j} + {b}_{j} ) \qquad ( 1 )$$

In various examples, any of a variety of feature encoders are employed (e.g., by the feature encoding module 120, etc.), for example, deep residual feature encoders such as a deep residual CNN (e.g., ResNet-50, etc.) truncated after the third residual block and pretrained on natural images (e.g., ImageNet, etc.) for examples with 2D patches, a spatiotemporal CNN (e.g., with a ResNet-50 backbone, etc.) pretrained on action recognition videos (e.g., Kinetics-400, etc.) for examples with 3D patches, etc. In the 2D and 3D prototypes, K=1024, although in some 2D and/or 3D examples K has greater or lesser values (e.g., depending on the feature encoder, etc.). The spatiotemporal CNN performs consistently well for both OTLS and microCT volumetric images, although a variety of other feature encoders are employed in various examples, with the performance of different feature encoders varying depending on the scenario in which the clinical endpoint prediction is made (e.g., the tissue and/or the morphological features that are more or less predictive in connection the potential clinical endpoints, etc.).

Because most feature encoders take three-channel red/green/blue (RGB) inputs, various examples emulate the setting by replicating channel information. Alternatively, by relying on algorithms or DL frameworks for false-coloring, a single-channel (e.g., microCT) or dual-channel (e.g., OTLS) image can be converted to display a three-channel image, similar to typical histopathology images. In one example, for dual-channel OTLS data, the nuclear channel data is replicated across the first two channels and the eosin channel is set as the third. In another example, for single-channel microCT data, the data is replicated across all three channels. For the feature encoding step, various batch sizes are used in various examples, which can vary based on the patches (e.g., the size of the patches, whether patches are 2D or 3D, etc.). Prototypes used a batch size of 500 for 2D patches and 100 for 3D patches.

Generation of the compressed features zⱼ∈ [symbol] ^{K′} from patches varies based on the feature encoder employed. As a first example utilizing a CNN-based feature encoder, the feature encoder outputs intermediate features (e.g., generated via the feature encoding module 120, etc.) that are 3-dimensional for a 2D patch (K, Ñ,\tilde{W}) and 4-dimensional for a 3D patch (K, \tilde{D}, Ñ, \tilde{W}), where \tilde{D}, \tilde{H}, \tilde{W} correspond to the down-sampled depth, height, and width dimensions, respectively. The intermediate features are compressed (e.g., by the feature encoding module 120, etc.) to one-dimensional feature hⱼ∈ [symbol] ^{K} with adaptive average-spatial pooling operation and subsequently to zⱼ∈ [symbol] ^{K′} with the fully-connected network. As a second example utilizing a ViT-based feature encoder, the Classify (CLS) token output of the VIT is treated as hⱼ∈ [symbol] ^{K} and subsequently compressed to zⱼ∈ [symbol] ^{K′} with the fully-connected network.

The patching (e.g., via the image processing module 118, etc.) and feature encoding (e.g., via the feature encoding module 120, etc.) operations result in a collection of K′ (e.g., 256, etc.)—dimensional features (e.g., also referred to as instances)

$${\{ {z}_{j} \}}_{j = 1}^{J} ,$$

constituting the volume with a single patient-level supervisory label. The

$${\{ {z}_{j} \}}_{j = 1}^{J}$$

features are used to train various examples using multiple instance learning (MIL) and/or to generate clinical endpoint predictions via trained examples. In various examples, MIL, a type of weakly-supervised learning, is used for training due to the substantial size of the input (e.g., number of patches) in comparison to the supervisory label. Various examples employ an attention-based aggregation module (e.g., via the feature aggregation module 122, etc.), for example, a lightweight attention network that learns to automatically compute an importance score of each patch feature and aggregates by weighted-averaging the features to form a single volume-level feature. In various examples, the attention network includes three sets of parameters V∈ [symbol] ^{K″×K′} (where K″<K′, e.g., K″=64 and K′=256, etc.), U∈ [symbol] ^{K″×K′}, and W∈ [symbol] ^{1×K″} The attention network assigns an importance score aⱼ∈[0,1] to feature zⱼ, as in equation (2):

$${a}_{j} = \frac{exp ( W ( tanh ( {Vz}_{j} ) ⊙ sigm ( {Uz}_{j} ) ) )}{{\sum }_{{j}^{′} = 1}^{J} exp ( W ( tanh ( V {z}_{{j}^{′}} ) ⊙ sigm ( {Uz}_{{j}^{′}} ) ) )} , \qquad ( 2 )$$

with tanh and sigm denoting the hyperbolic tangent and sigmoid function, respectively, and ⊙ denoting the element-wise multiplication (Hadamard product) operation. A high score (aⱼ close to 1) indicates that the corresponding patch is very relevant for sample-level prediction, while a low score (aⱼ close to 0) indicates little to no prognostic value. Based on aⱼ and zⱼ, the volume level feature zᵥₒₗᵤₘₑ is computed per equation (3):

$${z}_{volume} = {\sum }_{j = 1}^{J} {a}_{j} {z}_{j} ∈ {ℝ}^{{K}^{′}} . \qquad ( 3 )$$

While this explanation is centered around an attention-based aggregation example, the volume level feature can be computed with any aggregation approach, from simple averaging to Transformer-based self-attention.

In various examples, the volume level feature is fed into a final classification layer (e.g., implemented by the feature aggregation module 122, etc.) to generate a prediction (e.g., probability for a clinical endpoint, such as association with a high-risk group, etc.). In some examples, the final classification layer is parametrized by W_{cls}∈ [symbol] ^{1×K′} and bias b_{cls}∈ [symbol] , resulting in a probability for a given outcome (e.g., the high-risk group, etc.) p∈[0,1] per equation (4):

$$p = sigm ( {W}_{cls} {z}_{volume} + {b}_{cls} ) . \qquad ( 4 )$$

In various examples, one or more techniques (e.g., saliency mapping, which in some examples are generated via integrated gradient techniques, etc.) are employed to assess the relationship between an input (e.g., the set of instance features

$${\{ {h}_{j} \}}_{j = 1}^{J} ,$$

etc.) and the corresponding prediction (e.g., the probability p, etc.). As one example, integrated gradient (IG) techniques are employed to assign an IG score for each input (e.g., hⱼ, etc.), signifying the strength of each input's influence on the prediction, with the sign of the score indicating the direction of influence. For example, in a scenario where the prediction is a prognosis and p is the probability for the high-risk group, positive IG values increase the risk (e.g., unfavorable prognosis) and negative IG values decrease the risk (e.g., favorable prognosis), while IG values close to 0 have little or no prognostic influence (in examples where p is the probability for the low-risk group, the significance of positive and negative IG values is reversed).

In one example, where F denotes the sequence(s)/module(s) that map

$${\{ {h}_{j} \}}_{j = 1}^{J}$$

to p as

$$p = F ( {\{ {h}_{j} \}}_{j = 1}^{J} )$$

(e.g., the sequence of the fully-connected layer for the feature encoder (e.g., implemented via the feature encoding module 120, etc.), the attention aggregation, and the classification (e.g., both implemented via the feature aggregation module 122, etc.), M is the total number of IG interpolation steps, and h_{j,k} is the k^{th} element of the feature hⱼ, the IG score for hⱼ is given by equation (5):

$$IG ( {h}_{j} ) = {\sum }_{k = 1}^{K} {h}_{j , k} \times \frac{1}{M} {\sum }_{m = 1}^{M} \frac{\partial F ( {\{ \frac{m}{M} \cdot {h}_{j} \}}_{j = 1}^{J} )}{\partial {h}_{j , k}} , \qquad ( 5 )$$

assuming a zero feature baseline. In various such examples, once the IG scores are computed for the patch features of a volumetric image (e.g., of the volumetric image(s) 104, etc.), the negative IG values are normalized to [−1,0] and the positive IG values are normalized to (0,1], to ensure the sign and the influence of a patch do not get flipped.

Based on the computed saliency values (e.g., IG scores, etc.), various examples generate saliency maps, which can depend on the imaging technique of the volumetric image and/or the specific example. As one example for an OTLS volumetric image, false coloring based on the physics model (the Beer-Lambert law for absorption of light) of the dual-channel information of the raw OTLS data is used to generate a hematoxylin and eosin (H&E)-stained appearance.

In various examples, fine-grained 3D heatmaps are generated by using cuboid patches with partial overlap to reduce blocky effects. The extent of overlap can vary between examples and for different dimensions (e.g., one example used a 75% overlap in the 2D plane dimensions and 50% overlap along the depth dimension, etc.), with the extent of overlap providing a tradeoff where more fine-grained variation involves increased blocky effects. To compute an IG score (e.g., or other saliency value, etc.) for a given region, the raw IG scores (etc.) prior to normalization of all the patches covering the region are accumulated and divided by the number of overlapping patches to generate a combined IG score (etc.) for the given region. The combined IG scores (etc.) are normalized similarly to other IG scores (etc.). Based on the normalized IG scores (etc.), a coloring scheme (e.g., a coolwarm colormap with red and blue colors indicating positive and negative IG scores (etc.), respectively, etc.) is applied to the normalized IG scores (etc.), and overlaid with partial transparency (e.g., 0.4, although greater or lower values are used in other examples, e.g., any value between 0 and 1, 0.2-0.6, 0.3-0.5, etc.) on the raw volumetric image.

Various training and evaluation techniques are employed in connection with various examples, and techniques and associated parameters can depend on various factors, such as the nature of the tissue sample captured in the volumetric image(s), the imaging modality/modalities on which the image is trained, the nature of the clinical endpoint prediction, etc. Examples of parameters and techniques that vary between examples include: the number of training epochs (e.g., 50 epochs was used in connection with prototypes, etc.), the initial learning rate (e.g., 2×10⁻⁴ was used in connection with prototypes, etc.), the learning schedule (e.g., a cosine decay scheduler was used in connection with prototypes, etc.), the optimizer and associated parameters (e.g., the AdamW optimizer with default parameters of 61=0.9, β2=0.999, and weight decay of 5×10⁻⁴ was used in connection with prototypes, etc.), the batch size (e.g., the prototypes were trained based on a mini-batch size of 1 patient sample, pooling together patches across samples if multiple tissue samples existed for the patient), etc. Additionally, some examples employ gradient accumulation over two or more batches for training stability (e.g., prototypes employed a gradient accumulation of 10 training samples, etc.). The selection of patches for training within volumetric images (e.g., how many patches to use, how to select the patches to use, etc.) can also vary between examples. For example, prototypes randomly sampled 50% of the patches of each volumetric image as a means of data augmentation to prevent overfitting and inject diversity into training samples, and sampled 50% of the patches per plane or cuboid to ensure all depths were equally accounted for. Additional potential variations between examples include quantity, placement, and dropout probabilities of dropout layers (e.g., the prototypes employed a heavy dropout of p=0.5 after each fully-connected layer, etc.); other data augmentation schemes applied to patches (e.g., rotation, intensity jittering, etc., in addition to random sampling of patches); loss function (e.g., prototypes used the binary cross-entropy loss), etc.

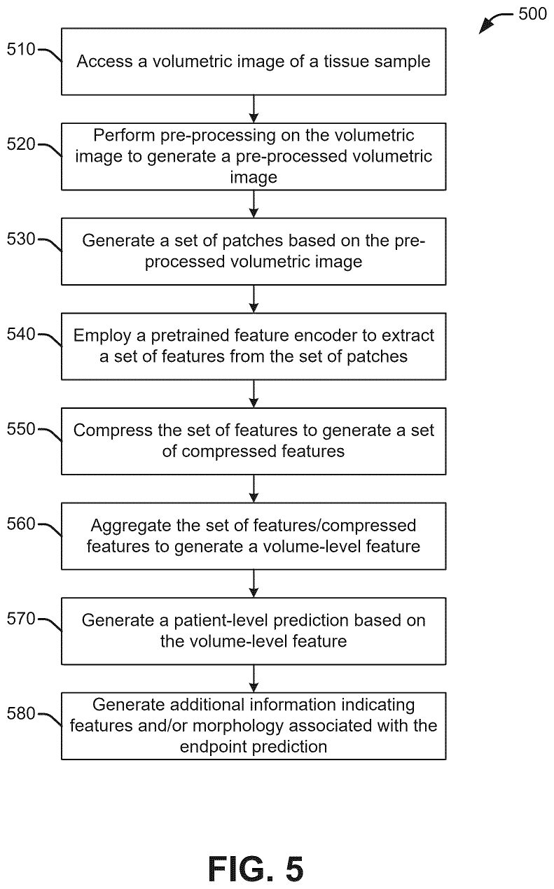

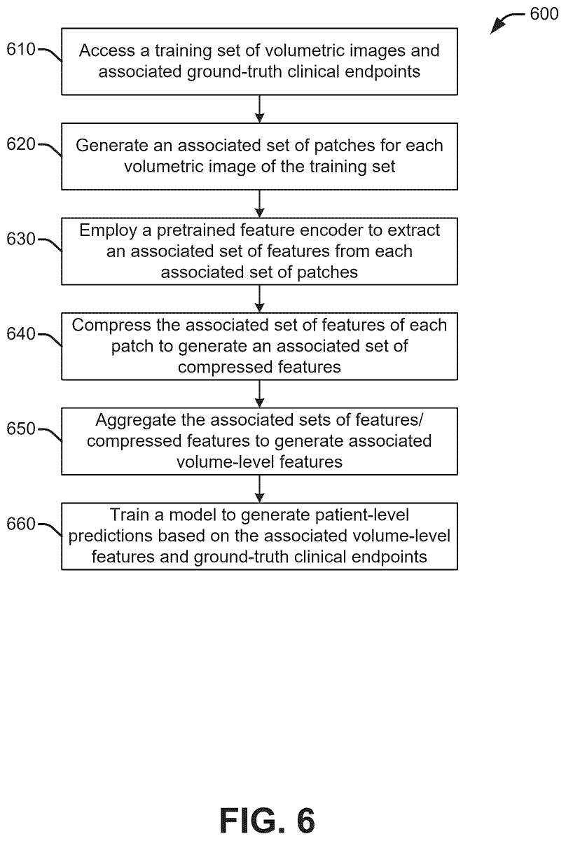

In view of the foregoing structural and functional features described above, example methods will be better appreciated with reference to FIGS. 5-6. While, for purposes of simplicity of explanation, the example methods of FIGS. 5-6 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

Referring to FIG. 5, illustrated is a flow diagram of a method 500 for generating a clinical endpoint prediction based on a volumetric image of a tissue sample. In other examples, the blocks of the example method 500 are a set of machine-readable instructions on a non-transitory machine-readable medium or are a set of operations performed by a processor executing machine-readable instructions as the operations.

At block 510, method 500 includes accessing a volumetric image (e.g., of the volumetric image(s) 104, etc.) of a tissue sample, for example, a raw volumetric image, a pre-processed volumetric image in examples omitting block 520, etc. In various examples, the volumetric image is obtained via any of a variety of imaging modalities, such as OTLS, microCT, etc.

At block 520, method 500 includes performing pre-processing (e.g., via the image processing module 118, etc.) on the volumetric image to generate a pre-processed volumetric image. In various examples, any of a variety of pre-processing techniques are employed, including tissue segmentation, segmenting the volumetric image into a stack of planes or cuboids, etc.

At block 530, method 500 includes generating (e.g., via the image processing module 118, etc.) a set of patches (e.g., 2D patches, 3D patches, etc.) based on the volumetric image (e.g., as pre-processed, etc.). In some examples, the set of patches includes patch-sized portions of the volumetric image containing tissue or containing greater than a threshold amount or percentage of tissue.

At block 540, method 500 includes employing (e.g., via the feature encoding module 120, etc.) a pretrained feature encoder to extract a set of features from the set of patches (e.g., a feature vector from each patch of the set of patches, etc.). Various examples employ different feature encoders, such as a 3D CNN, a 3D Vision Transformer, etc.

At block 550, method 500 includes compressing (e.g., via the feature encoding module 120, etc.) the set of features to generate a set of compressed features. In some examples, a domain-adapted shallow, fully-connected network is employed to generate the set of compressed features.

At block 560, method 500 includes generating a volume-level feature associated with the volumetric image via aggregation (e.g., via the feature aggregation module 122, etc.) based on the set of features (e.g., aggregating the set of compressed features or aggregating the set of features in examples omitting block 550). In various examples, the aggregation is based on assigning a weight to a given feature of the set of features, where the assigned weight is based on the importance of the given feature towards contributing to the volume-level feature to render a clinical endpoint prediction.

At block 570, method 500 includes generating (e.g., via the feature aggregation module 122, etc.) a clinical endpoint (e.g., patient-level) prediction based on the volume-level feature. Depending on the example, the prediction is one of a prognosis, a diagnosis, etc.

At block 580, method 500 includes generating (e.g., via the prediction interpretation module 124, etc.) additional information indicating features and/or morphology associated with the clinical endpoint prediction. In various examples, the additional information includes a saliency heatmap that is color-coded to indicate the extent to which various regions of the volumetric image contribute (e.g., positively or negatively, etc.) to the clinical endpoint prediction.

Referring to FIG. 6, illustrated is a flow diagram of a method 600 for training an artificial intelligence (AI) model to generate a clinical endpoint prediction based on a volumetric image of a tissue sample. In other examples, the blocks of the example method 600 are a set of machine-readable instructions on a non-transitory machine-readable medium or are a set of operations performed by a processor executing machine-readable instructions as the operations.

At block 610, method 600 includes accessing a training set of volumetric images (e.g., of the volumetric image(s) 104, etc.) of tissue samples and associated ground truth clinical endpoints, for example, raw volumetric images that are preprocessed at block 610, pre-processed volumetric images, etc. In various examples, the volumetric images are obtained via any of a variety of imaging modalities, such as OTLS, microCT, etc.

At block 620, method 600 includes generating (e.g., via the image processing module 118, etc.) an associated set of patches (e.g., 2D patches, 3D patches, etc.) based on each volumetric image (e.g., as pre-processed, etc.) of the training set. In some examples, the set of patches includes patch-sized portions of the volumetric image containing tissue or containing greater than a threshold amount or percentage of tissue.

At block 630, method 600 includes employing (e.g., via the feature encoding module 120, etc.) a pretrained feature encoder to extract an associated set of features from each associated set of patches (e.g., a feature vector from each patch of the associated set of patches, etc.). Various examples employ different feature encoders, such as a 2D/3D CNN, a 2D/3D Vision Transformer, etc.

At block 640, method 600 includes compressing (e.g., via the feature encoding module 120, etc.) each associated set of features to generate an associated set of compressed features. In some examples, a domain-adapted shallow, fully-connected network is employed to generate the associated sets of compressed features.

At block 650, method 600 includes generating an associated volume-level feature for each volumetric image of the training set via aggregation (e.g., via the feature aggregation module 122, etc.) based on the associated set of features (e.g., aggregating the associated set of compressed features or aggregating the set of features in examples omitting block 550). In various examples, the aggregation is based on assigning a weight to a given feature of the set of features, where the assigned weight is based on the importance of the given feature towards contributing to the volume-level feature to render a clinical endpoint prediction.

At block 660, method 600 includes training an AI model to generate (e.g., via the feature aggregation module 122, etc.) clinical endpoint (e.g., patient-level, etc.) predictions from volume-level features based on the associated volume-level features and ground truth clinical endpoints for the volumetric images of the training set. Depending on the example, the prediction is one of a prognosis, a diagnosis, etc.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Also as used herein, the term “set” means one or more elements (e.g., where the elements can be anything, such as datasets, nodes, relationships, etc.), and a “subset” of a set A refers to any set B where every element of set B is an element of set A (note that every set A is a subset of itself, as every element of set A is an element of set A). Additionally, where the disclosure or claims recite “a,” “an,” “a first,” or “another” element, or the equivalent thereof, it should be interpreted to include one or more than one such element, neither requiring nor excluding two or more such elements.

In this description, unless otherwise stated, “about,” “approximately” or “substantially” preceding a parameter means being within +/−10 percent of that parameter. Modifications are possible in the described embodiments, and other embodiments are possible, within the scope of the claims.

What is claimed is:

1. A non-transitory machine-readable medium having machine executable instructions for a physiological signal reconstruction system that cause a processor core to execute operations, the operations comprising:
generating a set of patches from a volumetric image of a tissue sample;
employing a pretrained feature encoder to extract a set of features from the set of patches;
generating a volume-level feature associated with the volumetric image via an aggregation based on the set of features; and
generating a clinical endpoint prediction based on the volume-level feature.

2. The non-transitory machine-readable medium of claim 1, wherein the operations further comprise compressing the set of features to generate a set of compressed features, wherein the aggregation comprises a weighted averaging of the set of compressed features.

3. The non-transitory machine-readable medium of claim 2, wherein the weighted average is based on a set of weightings assigned to the set of compressed features, wherein a weighting of the set of weightings is assigned to a compressed feature of the set of compressed features based on an importance determined for the compressed feature in connection with the clinical endpoint prediction.

4. The non-transitory machine-readable medium of claim 2, wherein the set of compressed features are generated via a fully connected network.

5. The non-transitory machine-readable medium of claim 1, wherein the set of patches comprises a set of three-dimensional (3D) patches.

6. The non-transitory machine-readable medium of claim 1, wherein the pretrained feature encoder is a three-dimensional (3D) feature encoder.

7. The non-transitory machine-readable medium of claim 1, wherein the operations further comprise generating a saliency heatmap based on the set of features, wherein the saliency heatmap visually represents the importance of regions of the volumetric image to the clinical endpoint prediction.

8. The non-transitory machine-readable medium of claim 1, wherein the operations further comprise performing a pre-processing on the volumetric image, wherein the set of patches are generated from the volumetric image after the pre-processing.

9. The non-transitory machine-readable medium of claim 8, wherein the pre-processing comprises a tissue segmentation.

10. The non-transitory machine-readable medium of claim 9, wherein the set of patches comprises patches having greater than a threshold amount of tissue based on the tissue segmentation.

11. The non-transitory machine-readable medium of claim 9, wherein the clinical endpoint prediction is generated via an artificial intelligence (AI) model trained on a set of training volumetric images, wherein a training volumetric image of the set of training volumetric images is associated with an imaging modality and the volumetric image is associated with the imaging modality.

12. A non-transitory machine-readable medium having machine executable instructions for training a physiological signal reconstruction system that cause a processor core to execute operations, the operations comprising:
accessing a training set comprising a set of volumetric images and a set of ground-truth clinical endpoints;
generating patches from the set of volumetric images, wherein an associated set of patches is generated from a volumetric image of the set of volumetric images;
employing a pretrained feature encoder to extract features from the patches, wherein an associated set of features is extracted from the associated set of patches;
generating a set of volume-level features associated with the set of volumetric images via an aggregation based on the features, wherein an associated volume-level feature of the set of volume-level features is generated based on the associated set of features; and
training an artificial intelligence (AI) model based on the set of volume-level features and the set of ground-truth clinical endpoints, wherein the AI model is trained based on the associated volume-level feature and a ground-truth clinical endpoint of the set of ground truth-clinical endpoints, and the ground-truth clinical endpoint is associated with the volumetric image.

13. The non-transitory machine-readable medium of claim 12, wherein the operations further comprise compressing the features to generate compressed features, wherein an associated set of compressed features is generated from the associated set of features, and the associated volume-level feature is based on a weighted average of the associated set of compressed features.

14. The non-transitory machine-readable medium of claim 12, wherein the compressed features are generated via a fully connected network.

15. The non-transitory machine-readable medium of claim 12, wherein the patches comprises three-dimensional (3D) patches.

16. The non-transitory machine-readable medium of claim 12, wherein the pretrained feature encoder is a three-dimensional (3D) feature encoder.

17. A method, comprising:
generating a set of patches from a volumetric image of a tissue sample;
employing a pretrained feature encoder to extract a set of features from the set of patches;
generating a volume-level feature associated with the volumetric image via an aggregation based on the set of features; and
generating a clinical endpoint prediction based on the volume-level feature.

18. The method of claim 17, further comprising compressing the set of features to generate a set of compressed features, wherein the aggregation comprises a weighted averaging of the set of compressed features.

19. The method of claim 18, wherein the weighted average is based on a set of weightings assigned to the set of compressed features, wherein a weighting of the set of weightings is assigned to a compressed feature of the set of compressed features based on an importance determined for the compressed feature in connection with the clinical endpoint prediction.

20. The method of claim 17, further comprising generating a saliency heatmap based on the set of features, wherein the saliency heatmap visually represents the importance of regions of the volumetric image to the clinical endpoint prediction.
